(19) United States
(12) Patent Application Publication
(10) Pub. No.: US 2017/0277831 A1
(43) Pub. Date: Sep. 28, 2017

(54) SYSTEM AND METHOD FOR GENERATING, STORING AND ACCESSING SECURED MEDICAL IMAGERY

(71) Applicants: Evan Ruff, Atlanta, GA (US); Greg Kolovich, Savannah, GA (US)

(72) Inventors: Evan Ruff, Atlanta, GA (US); Greg Kolovich, Savannah, GA (US)

(21) Appl. No.: 15/466,216

(22) Filed: Mar. 22, 2017

Related U.S. Application Data
(60) Provisional application No. 62/313,496, filed on Mar. 25, 2016.

(51) Int. Cl. G06F 19/00; A61B 6/00; G06F 21/60

(52) U.S. Cl. CPC G06F 19/321; G06F 21/602; G06F 19/328; A61B 6/485; G06Q 2220/10

(57) ABSTRACT

A system and method for generating, storing and accessing secure medical images uses public key cryptography, allowing users uses to capture, view and share images, as well as share the images with other authorized users and authorize other devices. The system is geared toward speed, security and portability of medical image processing. The method is capable of encrypting medical images and providing secured portable access to both the patient and physician. The invention is unique to the field of medicine by uploading fluoroscopic and digital pictures and video, in real time, to a medical record or patient-specific application.

### REFERENCE TO RELATED APPLICATIONS

This Application claims priority to U.S. Provisional Patent Application Ser. No. 62/313,496, filed Mar. 25, 2016, the entire content of which is incorporated herein by reference.

### FIELD OF THE INVENTION

This invention relates generally to secure file transfers and, in particular, to systems and methods for generating, storing and accessing secure medical imagery in real time, including static and dynamic fluoroscopic images of the human skeleton and high quality digital pictures and video of surgical anatomy and pathology.

### BACKGROUND OF THE INVENTION

Fluoroscopy is a process by which radiation emitted through a patient is converted into a radiographic image in real time, thus allowing for immediate depiction of skeletal anatomy. The physician uses these radiographic images to determine the presence or absence of pathology, the position and relation of radio-opaque objects within the patient (i.e., plates, screws or foreign bodies) or the quality of fracture reduction. Moreover, digital pictures and video are often needed to document relevant surgical anatomy or pathology.

Distal extremity surgery today, both hand/wrist and foot/ankle, requires use of intraoperative fluoroscopy and digital imagery for real time assessment of anatomy and pathology. A system by which both fluoroscopic and digital images can be securely processed, documented and made available to the patient and the physician via a patient-specific electronic application is paramount. These secured images can then be uploaded to a hospital-specific electronic medical record unique to that patient.

### SUMMARY OF THE INVENTION

Disclosed herein is a system and accompanying method that securely processes medical imagery in real time, including static and dynamic fluoroscopic and digital imagery for the purposes of aiding surgical treatment of the distal extremities. However, “medical image” should be taken to include any process of creating visual representations of the interior or exterior of a body for clinical analysis or medical intervention, as well as visual representations of organ function or tissues, as well as radiography, MRI, ultrasound, endoscopy, thermography, PET, SPECT, and so forth.

In accordance with the invention, all imagery is securely uploaded to a patient-specific electronic file application. All images are encrypted and secured in accordance with HIPAA Federal Regulations to protect the privacy and security of patient health information. Images can be decrypted only through the application of a private key, known only to authorized users. If a private key is lost, images can be re-encrypted through the application of a secured backup private key.

A method of generating, storing and accessing secure medical images comprising the steps of authorizing a user or a local computing device through a registration service, and generating a public/private encryption key pair for the authorized user or device. The public key is stored at a key service, with the private key being retained by the authorized user or device. Independently, the same or a different user is authorized to capture a medical image using a medical imaging device. The medical image is encrypted in the medical imaging device using the public key, and the encrypted image is stored at an image service. An authorized user or a device with a private key derived from the encryption key pair is authorized to decrypt and view the medical image on a display device.

In a preferred embodiment, a low-resolution, HIPPA compliant thumbnail corresponding to the encrypted medical image is also stored at the image service, which may be sent to the user or the device authorized to view an encrypted medical image in advance for image selection purposes. The system and method also accommodate an authorized user to authorize additional devices to view an encrypted image and/or share a secured image with another secured user or device.

A backup public/private encryption key pair may be generated for backup purposes, as well as a paper-based QR or other computer-readable code of the backup private encryption key. Such backup provisions may be used to restore a user's image library in the event of a lost or compromised system feature. As used herein, “local computing device” may be taken to mean is a smartphone, laptop, tablet or other portable electronic device, or desktop device with a high-resolution display for viewing purposes.

### BRIEF DESCRIPTION OF THE DRAWINGS

FIG. 1 is an overview of the system, illustrating the various components and the clusters involved;

FIG. 2 is an overview of an initial Key Registration Process, wherein a public/private key pair is generated on a user device and a backup key pair on a user device, as well as the archival of the backup private key and the syndication of the public keys;

FIG. 3 is an overview of the operational capture, encryption and upload process. The diagram shows the doctor, medical device, keys and cloud services working in tandem to secure medical images;

FIG. 4 is an overview of a typical image viewing process, including a user device using a private key to download an encrypted file;

FIG. 5 is an overview of an initial Device-to-Device Key Transfer Process, wherein an additional device is authorized to view images;

FIG. 6 is an overview of an Image Restoration Process in the event of compromise or loss of the operational public key; and

FIG. 7 is an overview of the Image Sharing Process in the case of a user desiring to share a secure image with another authorized party.

### DETAILED DESCRIPTION OF THE PREFERRED EMBODIMENTS

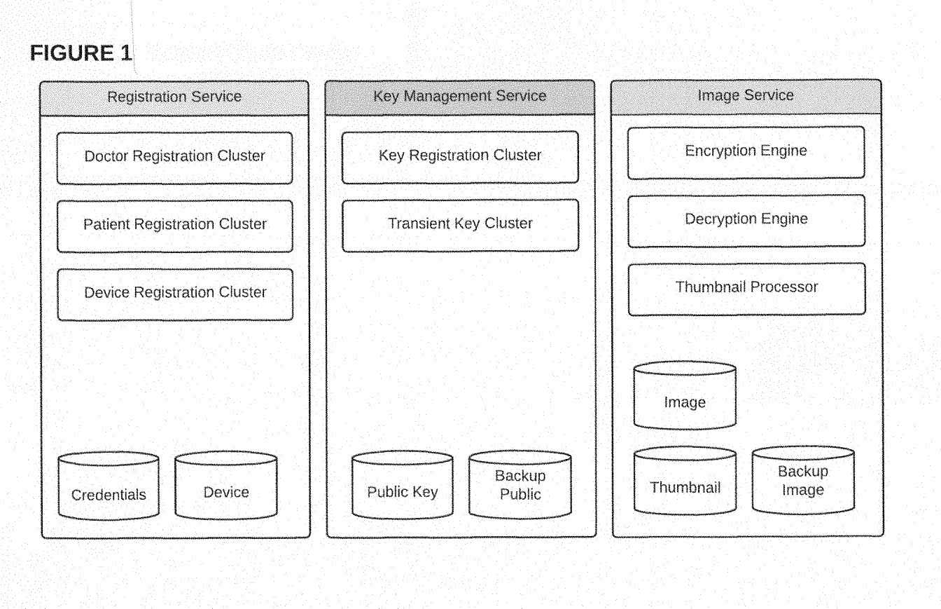

The Secured Medical Imagery (SMI) system described herein combines independent services that together create a secure, end-to-end medical imaging environment. Referring to FIG. 1, the first service is a registration service that stores the credentials of users and devices, and manages the registration of doctors, patients and the medical imaging devices themselves. The second service is a key management service. This system does not store any private key data, only public key data used to encrypt images. Rather, the service stores the public key and the backup public key for all known doctors and patients. The service can be queried to access these keys. The system also contains hardened, transient key functionality when it is necessary to restore a lost or compromised key. These private keys are never stored and are used only for the one-time decryption of images. The third service is an image service. This service stores the encrypted images, the backup encrypted images and the thumbnail previews of the images themselves.

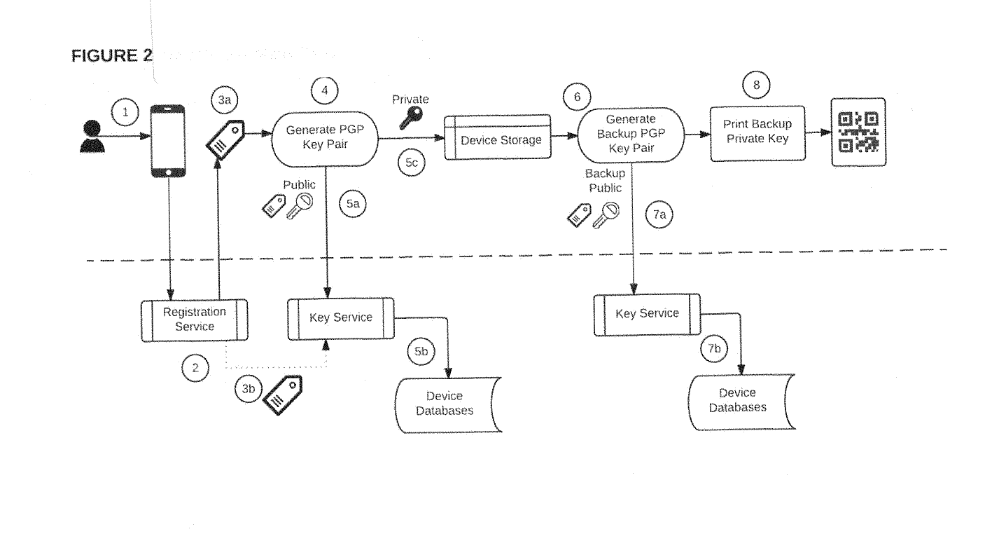

FIG. 2 illustrates the process by which devices and users create and share encryption keys for the medical imaging system. This system process operates as follows:
1. Using a local computing device, such as a laptop, smartphone or tablet, a user will login, sending credentials to a registration server.
2. The registration service will authorize the user through application of traditional username/password as well as a second factor of authentication, such as a text message, telephone call or rolling RSA-style key. These trust relationships may be long-lasting (i.e., weeks or months as desired).
3. After verifying the users' identity, the registration service will generate an authorization token. This token is only used to communicate with services for the immediate operation and expires quickly. The token is returned to the phone (3a) and sent to the Key Service (3b).
4. Upon receiving the authorization token, the local computing device will generate a public/private key pair.
5. The local computing device will submit the public key to the key service, along with the authorization token (5a). The key service will then use the authorization token to validate the request and store the token in the appropriate databases (5b). The local computing device will then store the private key in secure, local storage (5c).
6. The local computing device will generate a second public/private key pair for backup purposes.
7. The local computing device will submit the authorization token and the public key to the key service (7a) where it will be stored in the appropriate database (7b).
8. The local computing device will then generate a paper-based, QR code of the backup private key. The user must securely store this image for use in a disaster recovery scenario.

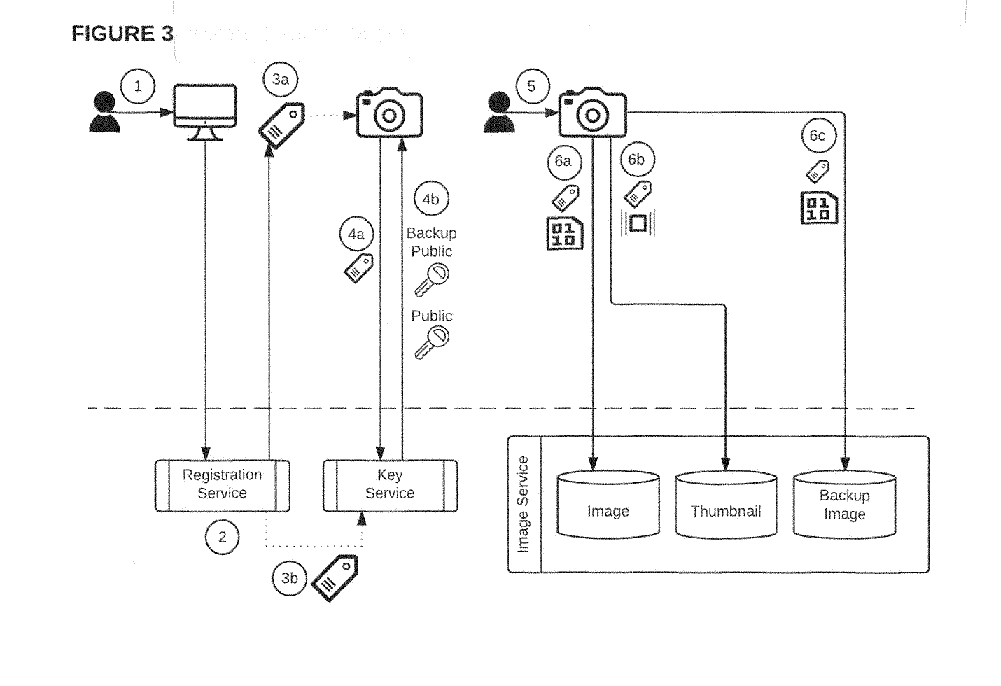

FIG. 3 illustrates the process by which the SMI will register a user, capture an image and then securely store that image in the cloud. This process works as follows:
1. Using a local computing device, such as a laptop, smartphone or tablet, a user will login, sending credentials to a registration server.
2. The registration service will authorize the user through application of traditional username/password as well as a second factor of authentication, such as a text message, telephone call or rolling RSA-style key.
3. After verifying the users' identity, the registration service will generate an authorization token. This token is only used to communicate with services for the immediate operation and expires quickly. The token is returned to the local computing device (3a) and sent to the Key Service (3b).
4. The local computing device transfers the authorization token to the medical imaging device. The medical imaging device then submits the authorization token to the Key Service (4a). The key service then validates the authorization token and returns two public keys: the user's public key and the user's backup public key.
5. The user will use the medical imaging device to capture an image.
6. Upon capture, the device will use the user's public key to encrypt the image and send it to the image service with the authorization token (6a). Additionally, the device will generate a small thumbnail preview of the image and send it to the image service (6b). Finally, the device will use the user's backup public key to encrypt the image and submit that, along with the authorization key, to the image service (6c).

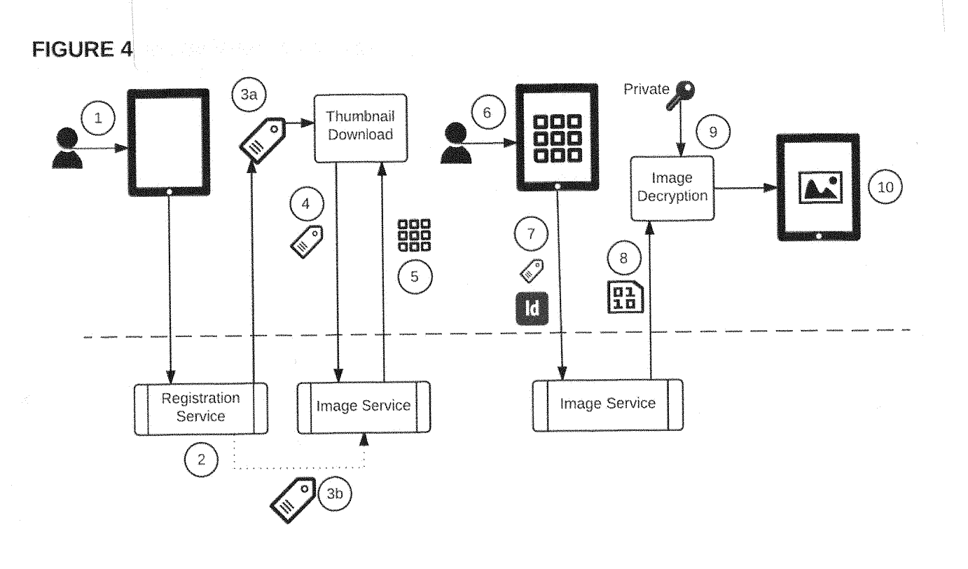

FIG. 4 illustrates the process by which a user will view images captured by SMI on a local storage devices. This system process as follows:
1. Using a local computing device, such as a laptop, smartphone or tablet, a user will login, sending credentials to a registration server.
2. The registration service will authorize the user through application of traditional username/password as well as a second factor of authentication, such as a text message, telephone call or rolling RSA-style key. These trust relationships may be long-lasting (i.e., weeks or months as desired).
3. After verifying the users' identity, the registration service will generate an authorization token. This token is only used to communicate with services for the immediate operation and expires quickly. The token is returned to the local computing device (3a) and sent to the Image Service (3b).
4. The local device will then submit the authorization token to the image service.
5. The image service will verify the authorization and return the image identifiers and thumbnail previews to the local device.
6. The user will select a thumbnail preview to view in full resolution.
7. The local device will submit the authorization token and the image identifier to the image service.
8. The image service will return the encrypted image to the local device.
9. Using the locally stored private key, the local device will decrypt the cipher text.
10. After decryption, the full resolution image will be displayed to the user.

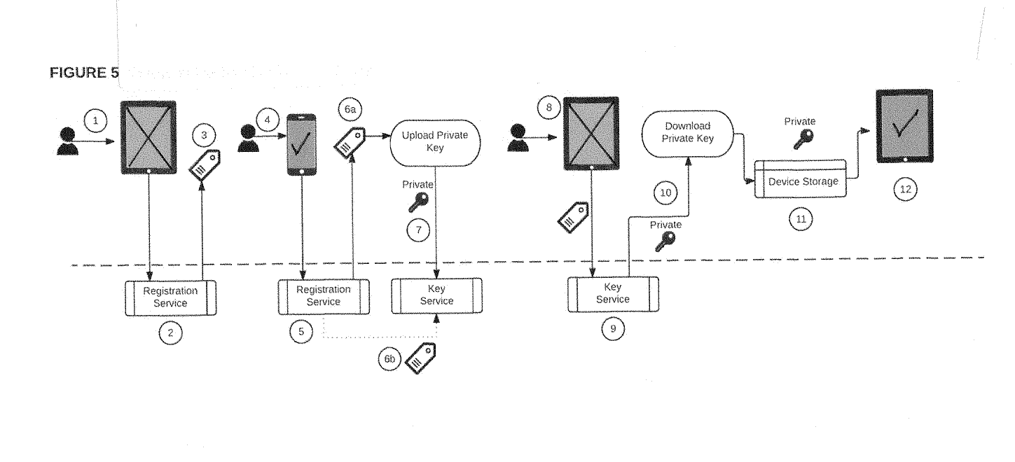

FIG. 5 illustrates the process by which a user will authorize additional devices to view the encrypted images stored in the SMI system. This process works as follows:
1. Using an unauthorized local computing device (depicted with an “X”), such as a laptop, smartphone or tablet, a user will notify the registration service of the intention to authorize the new device, sending credentials to a registration service.
2. The registration service will authorize the user through application of traditional username/password as well as a second factor of authentication, such as a text message, telephone call or rolling RSA-style key.
3. After verifying the users' identity, the registration service will generate an authorization token. This token is only used to communicate with services for the immediate operation and expires quickly. The token is returned to the unauthorized local computing device.
4. Using an authorized (depicted with a check mark) local computing device, such as a laptop, smartphone or tablet, a user will notify the registration service of the intention to allow the authorization of the new device, sending credentials to a registration service.
5. The registration service will authorize the user through application of traditional username/password as well as a second factor of authentication, such as a text message, telephone call or rolling RSA-style key.
6. After verifying the users' identity, the registration service will return the same authorization token as in step 3 to the authorized device (6a). It will also send this token to the key service (6b).
7. The authorized device will upload the private key as well as the authorization token to the key service.
8. Using the unauthorized device, the user will instruct the device to submit the authorization token to the key service.
9. The key service will return the private key to the device.
10. The unauthorized device will download the private key.
11. Upon receiving the private key, the unauthorized device will store the private key in the device's secure storage.
12. The previously unauthorized device is now able to view images.

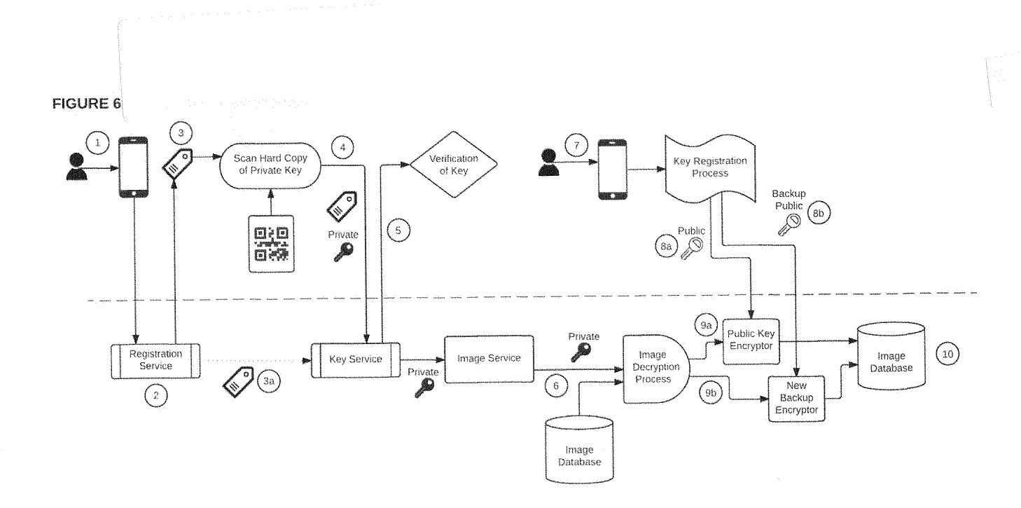

FIG. 6 illustrates the process by which the SMI system will restore a user's image library in the case of disaster, such as a lost or compromised device or the compromise of the entire user account. This process works as follows:
1. Using a local computing device, such as a laptop, smartphone or tablet, a user will login, sending credentials to a registration server.
2. The registration service will authorize the user through application of traditional username/password as well as a second factor of authentication, such as a text message, telephone call or rolling RSA-style key.
3. After verifying the users' identity, the registration service will generate an authorization token. This token is only used to communicate with services for the immediate operation and expires quickly. The token is returned to the local computing device (3a) and sent to the Key Service (3b).
4. Using the local device, the user will enter in the image of their hard copy backup private key using either a camera or a scanner. The local device will construct the backup private key from the image. The local device will submit the backup private key and the authorization key to the key service.
5. The key service will validate the backup key and notify the user that the restoration process is ready to begin.
6. The key service will transfer the private key to the image service. And use the private backup key to decrypt the user's backup image library within the image service itself.
7. The user will go through the standard Key Registration process, as illustrated in FIG. 2.
8. Upon completion of the Key Registration Process, both the new public (8a) and new public backup key (8b) will be transferred to the Image Service.
9. The image service will then encrypt the user's image library using both the new public key (9a) and the new public backup key (9b).
10. The encrypted image data will then be stored in the image service.

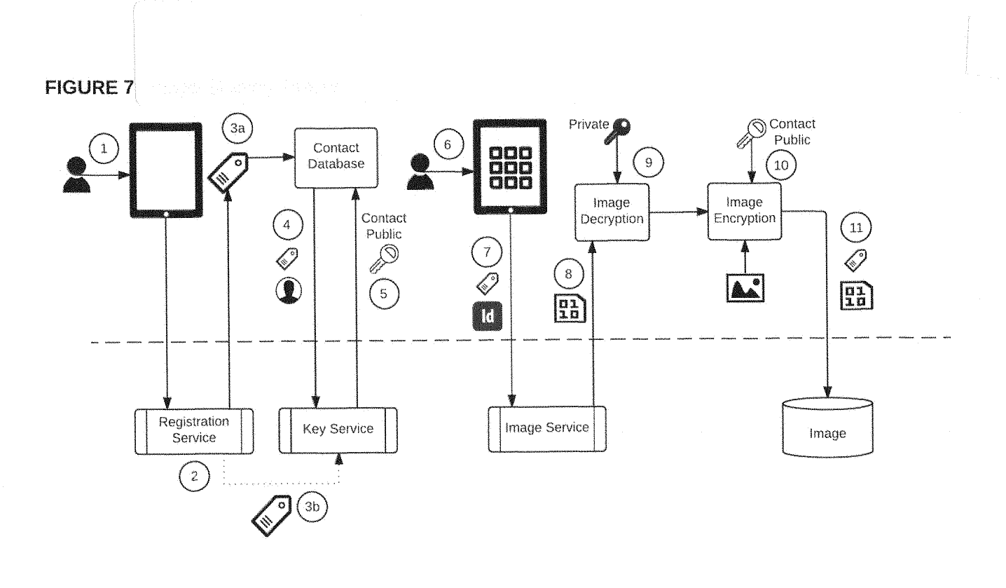

FIG. 7 illustrates the process by which a user can share a secured image with another secured user, such as a patient or another medical professional. The process works as follows:
1. Using a local computing device, such as a laptop, smartphone or tablet, a user will login, sending credentials to a registration server.
2. The registration service will authorize the user through application of traditional username/password as well as a second factor of authentication, such as a text message, telephone call or rolling RSA-style key.
3. After verifying the users' identity, the registration service will generate an authorization token. This token is only used to communicate with services for the immediate operation and expires quickly. The token is returned to the local computing device (3a) and sent to the Key Service (3b).
4. Using a contact database of other authorized SMI users, the user selects a given contact and submits the authorization token and user to the key service.
5. The key service verifies the authorization token and returns the public key of the desired contact.
6. The user then selects the images he wishes to share with the contact.
7. The local device submits the authorization token and the image identifier to the image service.
8. The image service returns the cipher text of the requested image.
9. The local device uses the locally stored private key to decrypt the image.
10. The decrypted image is then encrypted using the contact's public key.
11. The new cipher text image is submitted to the image service using the authorization token.

1. A method of generating, storing and accessing secure medical images, comprising the steps of:
(a) authorizing a user or a local computing device through a registration service;
(b) generating a public/private encryption key pair for the authorized user or device;
(c) storing the public key for the authorized user or device at a key service, while retaining the private key by the authorized user or device;
(d) authorizing a user to capture a medical image with a medical imaging device;
(e) encrypting the medical image in the medical imaging device with the public key stored at the key service;
(f) storing the encrypted medical image at an image service;
(g) authorizing a user or a device with a private key to view an encrypted medical image; and
(h) decrypting the image with the private key generated for the user or device, and displaying the decrypted medical image.

2. The method of claim 1, including the steps of:
storing a low-resolution thumbnail corresponding to the encrypted medical image stored at the image service;
sending one or more low-resolution thumbnail images from the image service to the user or the device authorized to view an encrypted medical image; and
upon selection of a low-resolution thumbnail image, decrypting and displaying the medical image corresponding to the thumbnail.

3. The method of claim 1, including the following steps to authorize an additional device to view an encrypted image:
receiving a request at the registration service from an unauthorized device to become authorized;
sending an authorization token from the registration service to the unauthorized device;
receiving a request at the registration service from an authorized user or device to register the unauthorized device;
sending the authorization token to the authorized user or device and to the key service; and
sending the private key of the authorized user or device from the key service to the unauthorized device, thereby authorizing the unauthorized device to view encrypted images stored at the image service.

4. The method of claim 1, including the following steps to share a secured image with another secured user or device:
authorizing a user or a local computing device through the registration service, and providing the user or local computing device with an authorization token;
selecting another authorized user from a database of authorized users, and submitting information identifying the other authorized user and the authorization token to the key service;
verifying the authorization token at the key service, and providing the user or a local computing device with the public key of the other authorized user;
submitting the authorization token to the image service along with information identifying at least one encrypted image to be shared;
delivering the encrypted image to the user or local computing device, and decrypting the encrypted image by the user or local computing device using their private key;
re-encrypting the decrypted image by the user or local computing device using the public key of the other authorized user provided to the user or local computing device by the key service; and
submitting the re-encrypted image to the image service using the authorization token for shared access by the other authorized user.

5. The method of claim 1, including the step of generating a backup public/private encryption key pair.

6. The method of claim 4, including the step of generating a paper-based QR or other computer-readable code of the backup private encryption key.

7. The method of claim 5, including the following steps to restore a user's image library:
authorizing a user or a local computing device through the registration service;
gathering an image of the paper-based computer-readable code by the user or local computing device, and generating the backup private encryption key based upon the gathered image;
validating the backup private key at the key service;
transferring the backup private key to the image service, and decrypting the user's image library at the image service using the backup private key;
generating a new public/private encryption key pair for the authorized user or device through the registration service;
storing the new public key for the authorized user or device at the key service, while retaining the new private key by the authorized user or device;
transferring the user's original stored public key and the new public key from the key service to the image service;
encrypting the user's image library using both the original stored public key and the new public key; and
storing the newly encrypted image library at the image service.

8. The method of claim 1, wherein any step involving authorizing a user includes one or more of the following:
a username and password,
a text message,
a telephone call, and
a rolling RSA-type key.

9. The method of claim 8, further including the step of providing an authorized user with a temporary authorization token.

10. The method of claim 1, wherein the local computing device is a smartphone, laptop, tablet or other portable electronic device.

11. The method of claim 1, wherein the medical image is a static or dynamic fluoroscopic image.

12. The method of claim 1, wherein the medical image is an anatomical or pathological image.
